(19) United States
(12) Patent Application Publication
(10) Pub. No.: US 2007/0197900 A1
(43) Pub. Date: Aug. 23, 2007

(54) Magnetic flow cytometer with SQUID microscopy

(75) Inventors: Franz Baudenbacher, Franklin, TN (US); Luis E. Fong, Nashville, TN (US); Eduardo Andrade Lima, Nashville, TN (US); David K. Schaffer, Nashville, TN (US); John Wikswo, Brentwood, TN (US)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: Vanderbilt University, Nashville, TN (US)

(21) Appl. No.: 11/604,018

(22) Filed: Nov. 22, 2006

Related U.S. Application Data
(60) Provisional application No. 60/738,814, filed on Nov. 22, 2005.

(51) Int. Cl. A61B 5/05

(52) U.S. Cl. 600/409

(57) ABSTRACT

A flow cytometer. In one embodiment, the flow cytometer has a microfluidic structure defining a channel with a periodically modulated path for transporting a stream of fluid with magnetic particles along the modulated path, and a superconducting quantum interference device (SQUID) sensor positioned over the microfluidic structure to define a detecting zone in the microfluidic structure for detecting magnetic signatures of a magnetic particle passing along the periodically modulated path through the detecting zone, where in use the stream of fluid with magnetic particles is regulated such that each magnetic particle passes singly along the periodically modulated path through the detecting zone.

### CROSS-REFERENCE TO RELATED PATENT APPLICATION

This application claims the benefit, pursuant to 35 U.S.C. §119(e), of U.S. provisional patent application Ser. No. 60/738,814, filed Nov. 22, 2005, entitled “A MAGNETIC FLOW CYTOMETER WITH SQUID MICROSCOPY,” by Franz Baudenbacher, Luis E. Fong, Eduardo Andrade Lima, David K. Schaffer, and John Wikswo, which is incorporated herein by reference in its entirety.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference. In terms of notation, hereinafter, “[n]” represents the nth reference cited in the reference list. For example, [9] represents the 9th reference cited in the reference list, namely, Holzer J. R., Fong L. E., Sidorov V. Y., Wikswo J. P., Baudenbacher F., Biophysical Journal 87, 4326 (2004).

### FIELD OF THE INVENTION

The present invention generally relates to a flow cytometer. More particularly, the present invention relates to a flow cytometer that has a microfluidic structure with a flow path modulation and a superconducting quantum interference device (SQUID) microscopy, and applications of same.

### BACKGROUND OF THE INVENTION

Magnetic microbeads are used in a great variety of biological and chemical assays [1, 2]. Typically separation techniques can not discriminate according to the magnetic moment and can only isolate magnetically tagged analytes from their non-magnetic counterparts. Many applications, especially cell sorting, would benefit from the ability to detect and discriminate a single moving magnetic bead. Several sensors technologies with magnetic field resolutions ranging from μT/Hz^{1/2} to several nT/Hz^{1/2} have been used to detect a static single magnetic bead: giant magnetoresistance (GMR) arrays [3], spin valve sensors [4], Hall sensors [5], Magnetic Force Microscopy [6], and AMR rings [7]. All these techniques require the magnetic particle to be directly placed on or bond to the surface of the sensor. However, in applications like flow cytometer it is not possible to bring the agglomerate of cell and magnetic label in such close proximity to the sensor. Therefore, sensors with a higher sensitivity are required to detect the agglomerates flowing pass the sensor. Superconducting quantum interference device (hereinafter “SQUID”) sensors provide higher sensitivity but have not been yet employed to detect a single magnetic bead. Microfluidics combined with SQUID microscopy may provide the sensitivity necessary to discriminate magnetic moments. This technology could have a large impact in high content, high throughput cell screening applications.

Therefore, a heretofore unaddressed need exists in the art to address the aforementioned deficiencies and inadequacies.

### SUMMARY OF THE INVENTION

In one aspect, the present invention relates to a flow cytometer. In one embodiment, the flow cytometer includes a microfluidic structure defining a channel with a periodically modulated path for transporting a stream of fluid with magnetic particles along the modulated path; and a SQUID sensor positioned over the microfluidic structure to define a detecting zone in the microfluidic structure for detecting magnetic signatures of a magnetic particle passing along the periodically modulated path through the detecting zone, where in use the stream of fluid with magnetic particles is regulated such that each magnetic particle passes singly along the periodically modulated path through the detecting zone. In one embodiment, the magnetic signatures of a magnetic particle comprise a temporal magnetic filed associated with the magnitude and the orientation of the magnetic moment of the magnetic particle passing through the detecting zone.

In one embodiment, the microfluidic structure is made of poly(dimethylsiloxane) (PDMS). The channel of the microfluidic structure has a cross-sectional dimension sized to accommodate a single magnetic particle.

The SQUID sensor comprises a directly-coupled low-temperature niobium based SQUID sensor. In one embodiment, the SQUID sensor comprises a washer-type SQUID sensor characterized with a SQUID inductance, L, a Josephson junction (JJ) critical current, I_{c}, a JJ self-capacitance, C, and a shunt resistance, Rₙ. The SQUID sensor in one embodiment is adapted such that when the SQUID operates at a temperature of about 4.2 K, the SQUID inductance L, the JJ critical current I_{c}, the JJ self-capacitance C, and the shunt resistance Rₙ satisfy the relationships of β_{c}=2πI_{c}Rₙ²C/φ₀≦0.7 and β_{L}=2LI_{c}/φ₀≅1, where φ₀ is a flux quantum of about 2×10⁻¹⁵ Wb.

The stream of fluid with magnetic particles comprises a stream of biological analytes, each biological analyte hosting a magnetic bead having a unique magnetic moment, where the magnetic bead comprises an amount of magnetic nanoparticles embedded in the core of the bead and magnetized such that the magnetic bead has a desired amount of remnant magnetization. In one embodiment, the magnetic bead has an analyte-specific surface coating. In another embodiment, the magnetic bead has an optical label including quantum dots. In one embodiment, the stream of biological analytes comprises one or more types of cells, where each cell is labeled with a cell-tracker dye. In another embodiment, the stream of biological analytes comprises one or more types of proteins.

The flow cytometer further includes a dewar having a tail portion configured to house the SQUID sensor in relation to the microfluidic structure such that there is a distance, d, between the SQUID sensor and the stream of fluid with magnetic particles passing along the microfluidic device structure. The flow cytometer also includes a window member having a first surface and an opposite, second surface defining a thickness, g, therebetween and positioned between the tail portion of the dewar and the microfluidic device structure, where the thickness g is less than the distance d and in a range of from about 1 μm to about 50 μm, preferably in a range of from about 5 μm to about 10 μm.

Furthermore, the flow cytometer includes an injecting member configured to introduce the stream of fluid with magnetic particles into the channel of the microfluidic structure, and means for driving the stream of fluid with magnetic particles to flow along the channel of the microfluidic structure. In one embodiment, the driving means comprises a pressurizer in communication with the channel of the microfluidic structure capable of applying a predetermined amount of pressure thereto. Moreover, the flow cytometer includes means for sorting each of the magnetic particles according to its detected magnetic signatures. Additionally, the flow cytometer may include a permanent magnet placed proximately to the channel of the microfluidic structure for polarizing each of the magnetic particles before it moves into the detecting zone.

In another aspect, the present invention relates to a flow cytometer. In one embodiment, the flow cytometer has a microfluidic structure having at least a first layer defining a fluidic channel, a second layer defining a control channel, and a membrane placed between the first layer and the second layer. The fluidic channel and the control channel are aligned to form one or more intersections therebetween, each intersection defining a valve such that the fluidic channel and the control channel are in communication with each other through the one or more valves. The fluidic channel of the microfluidic structure has a cross-sectional dimension sized to accommodate a single magnetic particle. The fluidic channel is configured to transport a stream of fluid with magnetic particles, while the control channel is configured to individually actuate and/or de-actuate each of the one or more valves. When one of the one or more valves is actuated, it allows a stream of fluid to flow from one side to the other side of the valve along the fluidic channel and vice versus. When one of the one or more valves is de-actuated, it allows no stream of fluid to flow from one side to the other side of the valve along the fluidic channel and vice versus. In one embodiment, the channel of the microfluidic structure is formed with a periodically modulated path. In another embodiment, the channel of the microfluidic structure is formed with a T-shape junction.

In one embodiment, the stream of fluid with magnetic particles comprises a stream of biological analytes, each biological analyte hosting a magnetic bead such that each of the magnetic particles has a unique magnetic moment.

The flow cytometer further has a SQUID sensor positioned over the microfluidic structure to define a detecting zone in the microfluidic structure for detecting magnetic signatures of a magnetic particle passing through the detecting zone. In one embodiment, the SQUID sensor comprises a directly-coupled low-temperature niobium based SQUID sensor. In another embodiment, the SQUID sensor comprises a washer-type SQUID sensor.

The flow cytometer also has a dewar having a tail portion configured to house the SQUID sensor in relation to the microfluidic structure such that there is a distance, d, between the SQUID sensor and the stream of fluid with magnetic particles passing along the microfluidic device structure.

Furthermore, the flow cytometer has a window member having a first surface and an opposite, second surface defining a thickness, g, therebetween and positioned between the tail portion of the dewar and the microfluidic device structure, where the thickness g is less than the distance d and in a range of from about 1 μm to about 50 μm.

Moreover, the flow cytometer has an injecting member configured to introduce the stream of fluid with magnetic particles into the channel of the microfluidic structure, means for driving the stream of fluid with magnetic particles to flow along the channel of the microfluidic structure, and a permanent magnet placed proximately to the channel of the microfluidic structure for polarizing each of the magnetic particles before it moves into the detecting zone.

Additionally, the flow cytometer has means for sorting each of the magnetic particles according to its detected magnetic signatures, where the sorting means comprises a controller in communication with the SQUID sensor and the one or more valves for receiving the detected magnetic signatures of each of the magnetic particles and generating a corresponding trigger signal for each of the magnetic particles to actuate and/or de-actuate each of the one or more valves, thereby sorting each magnetic particle into its designated port.

In yet another aspect, the present invention relates to a flow cytometer. In one embodiment, the flow cytometer includes a microfluidic structure having a channel and one or more valves formed on the channel, where the fluidic channel is configured to transport a stream of fluid with magnetic particles, and where when one of the one or more valves is actuated, it allows a stream of fluid to flow from one side to the other side of the valve along the fluidic channel and vice versus, and where when one of the one or more valves is de-actuated, it allows no stream of fluid to flow from one side to the other side of the valve along the fluidic channel and vice versus. Furthermore, the flow cytometer includes a SQUID sensor positioned over the microfluidic structure to define a detecting zone in the microfluidic structure for detecting magnetic signatures of a magnetic particle passing through the detecting zone. Moreover, the flow cytometer includes means for sorting each of the magnetic particles according to its detected magnetic signatures, where the sorting means comprises a controller in communication with the SQUID sensor and the one or more valves for receiving the detected magnetic signatures of each of the magnetic particles and generating a corresponding trigger signal for each of the magnetic particles to actuate and/or de-actuate each of the one or more valves, thereby sorting each magnetic particle into its designated port.

In a further aspect, the present invention relates to a method of detecting magnetic particles. In one embodiment, the method comprises the steps of: providing a microfluidic structure having a fluidic channel and a detecting zone defined with the fluidic channel; introducing a stream of fluid with magnetic particles into the fluidic channel; driving the stream of fluid with magnetic particles to flow along the fluidic channel, where the stream of fluid with magnetic particles is regulated such that each magnetic particle passes singly through the detecting zone; and detecting magnetic signatures of a magnetic particle passing through the detecting zone. In one embodiment, the detecting step is performed with a SQUID sensor that is positioned over the detecting zone such that there is a distance, d, between the SQUID sensor and the stream of fluid with magnetic particles passing through the detecting zone.

In one embodiment, the stream of fluid with magnetic particles comprises a stream of biological analytes, each biological analyte hosting a magnetic bead such that each of the magnetic particles has a unique magnetic moment. In one embodiment, the fluidic channel of the microfluidic structure has a periodically modulated path. In another embodiment, the fluidic channel of the microfluidic structure has a T-shape junction. In an alternative embodiment, the fluidic channel of the microfluidic structure has one or more valves, where when one of the one or more valves is actuated, it allows a stream of fluid to flow from one side to the other side of the valve along the fluidic channel and vice versus, and where when one of the one or more valves is de-actuated, it allows no stream of fluid to flow from one side to the other side of the valve along the fluidic channel and vice versus.

Furthermore, the method comprises the step of sorting each of the magnetic particles according to its detected magnetic signatures, where the sorting step comprises the steps of: receiving the detected magnetic signatures of each of the magnetic particles;

and generating a corresponding trigger signal for each of the magnetic particles to actuate and/or de-actuate each of the one or more valves, thereby sorting each magnetic particle into its designated port.

In yet a further aspect, the present invention relates to a method of discriminating and/or sorting biological analytes. In one embodiment, the method includes the steps of:

preparing a magnetically-labeled analyte sample; and providing a flow cytometer. The flow cytometer comprises a microfluidic structure defining a channel; and a SQUID sensor positioned over the microfluidic structure to define a detecting zone in the microfluidic structure for detecting magnetic signatures of a magnetic particle passing along the channel through the detecting zone. Furthermore, the method includes the steps of introducing the magnetically-labeled analyte sample into the channel of the microfluidic structure; and detecting magnetic signatures of each analyte of the magnetically-labeled analyte sample passing along the channel through the detecting zone so as to sort the magnetically-labeled analyte sample according to its detected magnetic signatures.

In one embodiment, the magnetically-labeled analyte sample comprises CD51 positive (CD51+) melanoma cells (m21) and CD51 negative (CD51−) melanoma cells (m21-L). The preparing step comprises the steps of: labeling each of the m21 cells with a red cell-tracker dye and each of the m21-L cells with a green cell-tracker dye, respectively; mixing the labeled m21 cells and the labeled m21-L cells to produce a cell mixture; incubating the cell mixture with an anti-CD51 antibody (Ab1) followed by magnetic beads coated with a secondary antibody (Ab2) so as to produce a magnetically-labeled cell sample, where Ab2 is specific to Ab1, whereby the magnetic beads are only bound to the m21 cells and free from Ab1; and purifying the magnetically-labeled analyte sample by magnetic bulk separation. The method further comprises the step of quantifying the number of red and green fluorescent cells.

In another embodiment, the magnetically-labeled analyte sample comprises human Th1/Th2 cytokines including interleukin (IL)-2, IL-4, IL-5, IL-6, IL-10, tumor necrosis factor (TNF) and interferon-γ IFN-γ. Accordingly, the preparing step comprises the step of incubating magnetic beads with a solution containing the human Th1/Th2 cytokines and secondary fluorescent antibodies.

In one aspect, the present invention relates to a vector microscope. In one embodiment, the vector microscope has three orthogonally oriented SQUID sensors, where the three SQUID sensors are mounted onto a tip of a sapphire cube, where in operation, the sapphire cube is diagonally aligned normal to a scanning plane. In one embodiment, the SQUID sensor comprises a directly-coupled low-temperature niobium based SQUID sensor. In another embodiment, the SQUID sensor comprises a washer-type SQUID sensor characterized with a SQUID inductance, L, a Josephson junction (JJ) critical current, I_{c}, a JJ self-capacitance, C, and a shunt resistance, Rₙ, where the SQUID sensor is adapted such that when the SQUID operates at a temperature of about 4.2 K, the SQUID inductance L, the JJ critical current I_{c}, the JJ self-capacitance C, and the shunt resistance Rₙ satisfy the relationships of β_{c}=2πI_{c}Rₙ²C/φ₀≦0.7 and β_{L}=2LI_{c}/φ₀≅1, where φ₀ is a flux quantum of about 2×10⁻¹⁵ Wb.

In another aspect, the present invention relates to a method of probing the mechanical properties and cell motility of single cells. In one embodiment, the method includes the steps of: (a) providing a cell sample containing cells and magnetic beads, each magnetic bead attached to a corresponding cell to form a cell-bead unit such that when the magnetic bead moves and/or rotates, the corresponding cell moves and/or rotates accordingly; (b) providing a microfluidic structure defining a detecting zone capable of trapping a single cell therein; (c) providing a vector microscope positioned proximately to the detecting zone of the microfluidic structure, where the vector microscope comprises three SQUID sensors orthogonally oriented for simultaneously measuring three orthogonal components of a magnetic field of a cell trapped in the detecting zone; (d) introducing the cell sample into the microfluidic structure, where the cell sample is regulated such that each cell-bead unit passes singly through the detecting zone; (e) applying a first magnetic field to the cell sample along a first direction, where the first magnetic field comprises a magnetic pulse having an amplitude adapted for saturating magnetic moments of the magnetic beads; (f) applying a second magnetic field to the cell sample along a second direction orthogonally to the first direction, where the second magnetic field comprises a uniform magnetic field adapted for creating a torque on the magnetic beads so as to cause them to rotate from a first orientation to a second orientation; (g) turning off the second magnetic field so as to allow the magnetic beads to recover from the second orientation to the first orientation; and (h) continuously measuring a transient magnetic field of the cell-bead unit in the detecting zone in steps (e)-(g), where the measured magnetic field is related to the angular rotation of the magnetic bead of the cell-bead unit and hence to the angular rotation of the corresponding cell, where the angular rotation of the corresponding cell varies with time and the strength of the second field, and is related to the mechanical properties and cell motility of the corresponding cell.

In one embodiment, each magnetic bead is embedded within a corresponding cell through phagocytosis or injection. In another embodiment, each magnetic bead is bound to the cell membrane of a corresponding cell by coupling of ligand-coated beads of specific cell membrane receptors.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment, and wherein:

FIG. 1 shows schematically a flow cytometer according one embodiment of the present invention: (a) a superimposed image of a microfluidic structure and a SQUID sensor, where P1, P2, P3, P4 and P5 are reference points along the magnetic bead trajectory, and (b) a partially cross-sectional view of the flow cytometer;

FIG. 2 shows schematically a directly-coupled low-temperature niobium based SQUID sensor utilized in a flow cytometer according to one embodiment of the present invention;

FIG. 3 shows measured time traces (a) and (b) of the magnetic field from two different magnetic particles moving through the microfluidic structure of the flow cytometer shown in FIG. 1;

FIG. 4 shows time traces of simulated magnetic fields and corresponding projected magnetic moments to model the experimental observations of single particles moving in the microfluidic structure of the flow cytometer shown in FIG. 1: (a) the magnetic moment of the moving particle being a fixed direction, (b) the angle between the magnetic moment of the particle and the tangent of the path of the particle remaining constant, (c) the angle between the magnetic moment of the particle and the tangent of the path of the particle remaining changes; and (d), (e) and (f) time traces of the z-component of the magnetic field corresponding to the configuration in (a), (b) and (c), respectively, where labels P1-P5 correspond to the reference points in FIG. 1.

FIG. 5 shows schematically a microfluidic valve formed in an intersection between a fluidic channel and a control channel, which is utilized in a flow cytometer according one embodiment of the present invention: (a) the microfluidic valve being actuated, and (b) the microfluidic valve being de-actuated;

FIG. 6 shows schematically a vector SQUID microscope according to one embodiment of the present invention: (a) a prototype sensor including a sapphire rod wound with Nb—Ti pickup coil, (b) a vector sensor comprising three, planar SQUID-bearing plates orthogonally mounted on the sapphire tip, and (c) a enlarged view of one of fractional turn SQUID sensors;

FIG. 7 shows schematically a single cell magnetic twisting cytometer according to one embodiment of the present invention, where a twisting field is applied and from the relationship between torque and angular rotation rheological properties can be extracted; and

FIG. 8 shows schematically a process of probing the viscoelastic properties of a tumor cell during extravasation induced by a chemokine in a microenvironment according to one embodiment of the present invention: (a), (b) and (c) changes of the tumor cell at different times.

### DETAILED DESCRIPTION OF THE INVENTION

The present invention is more particularly described in the following examples that are intended as illustrative only since numerous modifications and variations therein will be apparent to those skilled in the art. Various embodiments of the invention are now described in detail. Referring to the drawings, like numbers indicate like parts throughout the views. As used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which has no influence on the scope of the invention. Additionally, some terms used in this specification are more specifically defined below.

### Definitions

The terms used in this specification generally have their ordinary meanings in the art, within the context of the invention, and in the specific context where each term is used.

Certain terms that are used to describe the invention are discussed below, or elsewhere in the specification, to provide additional guidance to the practitioner in describing the apparatus and methods of the invention and how to make and use them. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term. Likewise, the invention is not limited to various embodiments given in this specification. Furthermore, subtitles may be used to help a reader of the specification to read through the specification, which the usage of subtitles, however, has no influence on the scope of the invention.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

As used herein, the term “cell” refers to the smallest structural unit of an organism that is capable of independent functioning, consisting of one or more nuclei, cytoplasm, and various organelles, all surrounded by a semipermeable cell membrane.

The term “cytokine”, as used herein, refers to a group of proteinaceous signaling compounds that, like hormones and neurotransmitters, are used extensively for inter-cell communication. Cytokines are produced by a wide variety of cell types (both haemopoietic and non-haemopoietic) and can have effects on both nearby cells or throughout the organism, sometimes strongly dependent on the presence of other chemicals and cytokines.

As used herein, the acronym “SQUID” refers to a superconducting quantum interference device that is an extremely sensitive magnetic flux-to-voltage transducer used to measure extremely small magnetic fields. The fundamental component of the SQUID is the Josephson junction, essentially two superconductors weakly coupled through a small insulating gap or constriction. The Josephson junction has unique electrical/magnetic properties and when incorporated into a superconducting loop forms a SQUID. There are two main types of SQUID: dc-SQUID and rf-SQUID. Rf-SQUIDs have only one Josephson junction whereas dc-SQUIDs have two or more junctions. A flux-locked loop is used in both cases to make the flux-to-voltage transduction linear. Typically, a superconducting pick-up coil is used to funnel flux into the SQUID loop to increase the SQUID's voltage response.

### OVERVIEW OF THE INVENTION

The present invention, among other things, discloses a flow cytometer having microfluidic structure combined with a SQUID microscopy, which provides the sensitivity necessary to discriminate magnetic moments as small as 10⁻¹⁸ Am²/Hz^{1/2}. This corresponds to magnetic moments typically carried by iron oxide or cobalt magnetic nanocrystals with a size on the order of tens of nanometers. Being able to use such small magnetic particles as labels with potentially thousands of distinguishable magnetic moments would not only have a large impact on high-content, high-throughput analyte screening applications but may also allow measurement of properties not measurable with conventional flow cytometers. The flow cytometer of the present invention can find widespread applications in a variety of fields, particularly in the fields of sorting magnetically labeled cells according to the type of surface receptor expression, and selectively extracting and isolating multiple unique cytokines from a standard mixture of human Th1/Th2 cytokines using magnetic beads (nanoparticles) coated with cytokine-specific antibodies and having different magnetic moments.

The description will be made as to the embodiments of the present invention in conjunction with the accompanying drawings 1-8.

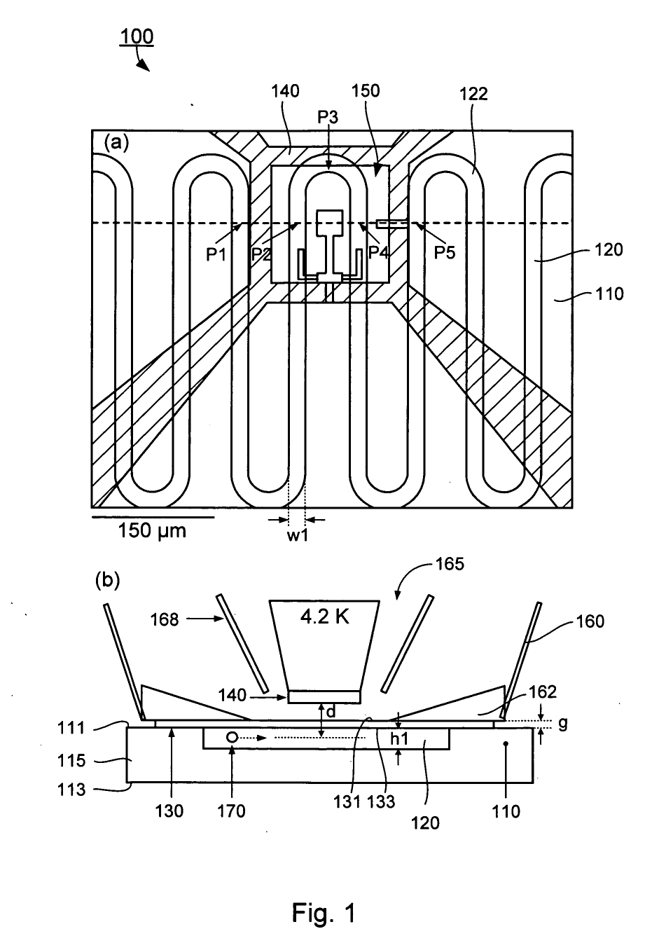

Referring to FIG. 1, a flow cytometer 100 is shown according to one embodiment of the present invention. In this embodiment, the flow cytometer 100 includes a microfluidic structure 110. The microfluidic structure 110 has a first surface 111 and an opposite, second surface 113 defining a body portion 115 therebetween. The body portion 115 of the microfluidic structure 110 defines a channel 120 proximate to the first surface 11 of the microfluidic structure 110. The channel 120 is formed with a periodically modulated path 122 for transporting a stream of fluid with magnetic particles along the modulated path 122. The channel has a width, w1, and a height, h1, where the width w1 and the height h1 are configured to be able to accommodate a stream of fluid with magnetic particles. The periodically modulated path 122 is formed in the form of serpentine. Other forms of the periodically modulated path, such as T-shape junctions, can be also used to practice the present invention. The flow cytometer 100 also includes a directly-coupled low-temperature niobium based SQUID sensor 140. The SQUID sensor 140 is housed in a vacuum space 165 of a dewar 160 and positioned over the microfluidic structure 110 to define a detecting zone 150 in the microfluidic structure 110 for detecting magnetic signatures of a magnetic particle 170 passing along the periodically modulated path 122 through the detecting zone 150. Additionally, the SQUID sensor 140 is surrounded with a radiation shield 168 inside the dewar 160. In this embodiment, the SQUID sensor 140 is positioned such that there is a distance, d, between the SQUID sensor 140 and a magnetic particle 170 passing along the periodically modulated path 122 through the detecting zone 150. Furthermore, a sapphire window 130 is positioned between the SQUID sensor 140 and the microfluidic structure 110 for separating the vacuum space from the room temperature sample particle in the stream of fluid with magnetic particles. The window 130 has a first surface 131 in contact with the tail portion 162 of the dewar 160 and an opposite, second surface 133 in contact with the first surface 111 of the microfluidic structure 110, defining a thickness, g, therebetween. The thickness g is less than the distance d and in a range of from about 1 μm to about 50 μm, preferably in a range of from about 5 μm to about 10 μm. Other types of window members such as a silicon nitride window can also be utilized to practice the present invention.

The stream of fluid with magnetic particles is regulated such that each magnetic particle passes singly along the periodically modulated path 122 through the detecting zone 150. Each magnetic particle is characterized with a unique magnetic moment. The magnetic particles can be cells, proteins of a living subject, or other particles.

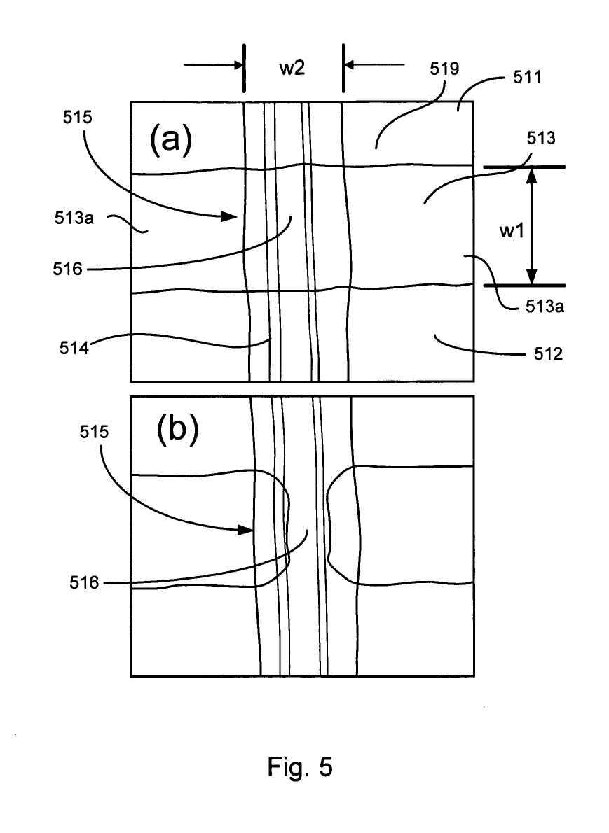

The microfluidic structure can be formed in other forms. For example, in one embodiment, the microfluidic structure includes a fluidic channel and one or more valves formed on the fluidic channel. The fluidic channel is configured to transport a stream of fluid with magnetic particles. When one of the one or more valves is actuated, it allows a stream of fluid to flow from one side to the other side of the valve along the fluidic channel and vice versus. When one of the one or more valves is de-actuated, it allows no stream of fluid to flow from one side to the other side of the valve along the fluidic channel and vice versus. The microfluidic structure may comprise a multilayer structure. For example, as shown in FIG. 5, the microfluidic structure has a first layer 511 defining a fluidic channel 513, a second layer 512 defining a control channel 514, and a membrane 519 placed between the first layer 511 and the second layer 512. The fluidic channel 513 and the control channel 514 are aligned to form one or more intersections 515 therebetween. Each intersection 515 defies a valve 516 such that the fluidic channel 513 and the control channel 514 are in communication with each other through the one or more valves 516. The control channel 514 is configured to individually actuate and/or de-actuate each of the one or more valves 516. The valve 516 shown in FIG. 5a is actuated so that a stream of fluid is allowed to flow through the actuated valve 516 from one side 513a to the other side 513b of the fluidic channel 513, and vice versus. The valve 516 shown in FIG. 5b is de-actuated so that no stream of fluid is allowed to flow through the actuated valve 516 from one side 513a to the other side 513b of the fluidic channel 513, and vice versus. In one embodiment, the fluidic channel 513 and the control channel 514 have a width, w1 and w2, respectively. In this embodiment, the flow cytometer may include a controller in communication with the SQUID sensor and the one or more valves for receiving the detected magnetic signatures of each of the magnetic particles and generating a corresponding trigger signal for each of the magnetic particles to actuate and/or de-actuate each of the one or more valves, thereby sorting each magnetic particle into its designated port.

In another aspect, the present invention relates to a method for identifying a moving magnetic particle. In one embodiment, the method includes the steps of forming a channel with a periodically modulated path, injecting a stream of magnetic particles into the channel so as to transport the stream of fluid with magnetic particles along the modulated path, where the stream of fluid with magnetic particles is regulated such that each magnetic particle passes singly along the periodically modulated path through a detecting zone, and detecting magnetic signatures of a magnetic particle passing along the periodically modulated path through the detecting zone so as to identify the moving magnetic particle. The channel is formed in a microfluidic structure, whereby the detecting zone is defined in the microfluidic structure. The detecting step is performed with a directly-coupled low-temperature niobium based SQUID sensor.

Yet another aspect of the present invention relates to a method of discriminating and/or sorting biological analytes. The method comprises the steps of preparing a magnetically-labeled analyte sample; providing a flow cytometer comprising: a microfluidic structure defining a channel; and a superconducting quantum interference device (SQUID) sensor positioned over the microfluidic structure to define a detecting zone in the microfluidic structure for detecting magnetic signatures of a magnetic particle passing along the channel through the detecting zone. The method further includes the steps of introducing the magnetically-labeled analyte sample into the channel of the microfluidic structure; and detecting magnetic signatures of each analyte of the magnetically-labeled analyte sample passing along the channel through the detecting zone so as to sort the magnetically-labeled analyte sample according to the detected magnetic signatures of each cell of the magnetically-labeled analyte sample.

In one embodiment, the magnetically-labeled analyte sample comprises CD51 positive (CD5 1+) melanoma cells (m21) and CD51 negative (CD51−) melanoma cells (m21-L), and the preparing step comprises the steps of: labeling each of the m21 cells with a red cell-tracker dye and each of the m21-L cells with a green cell-tracker dye, respectively; mixing the labeled m21 cells and the labeled m21-L cells to produce a cell mixture; incubating the cell mixture with an anti-CD51 antibody (Ab1) followed by magnetic beads coated with a secondary antibody (Ab2) so as to produce a magnetically-labeled cell sample, wherein Ab2 is specific to Ab1, whereby the magnetic beads are only bound to the m21 cells and free from Ab1; and purifying the magnetically-labeled analyte sample by magnetic bulk separation.

In another embodiment, the magnetically-labeled analyte sample comprises human Th1/Th2 cytokines including interleukin (IL)-2, IL-4, IL-5, IL-6, IL-10, tumor necrosis factor (TNF) and interferon-γ IFN-γ, and the preparing step comprises the step of incubating magnetic beads with a solution containing the human Th1/Th2 cytokines and secondary fluorescent antibodies.

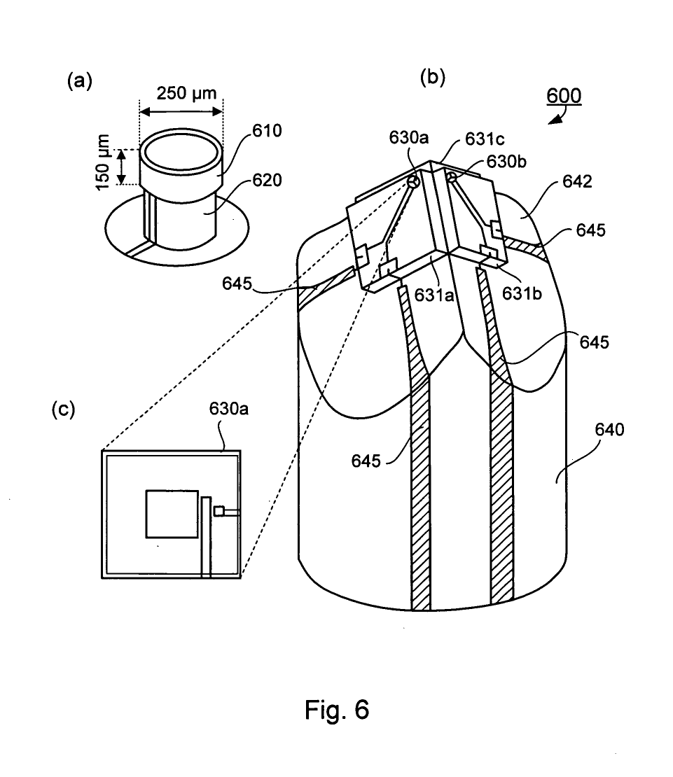

One aspect of the present invention relates to a vector microscope. The vector microscope 600 in one embodiment includes three orthogonally oriented SQUID sensors 630a, 630b and 630c, as shown in FIG. 6. The three SQUID sensors 630a, 630b and 630c are mounted onto a tip of a sapphire cube, where in operation, the sapphire cube is diagonally aligned normal to a scanning plane.

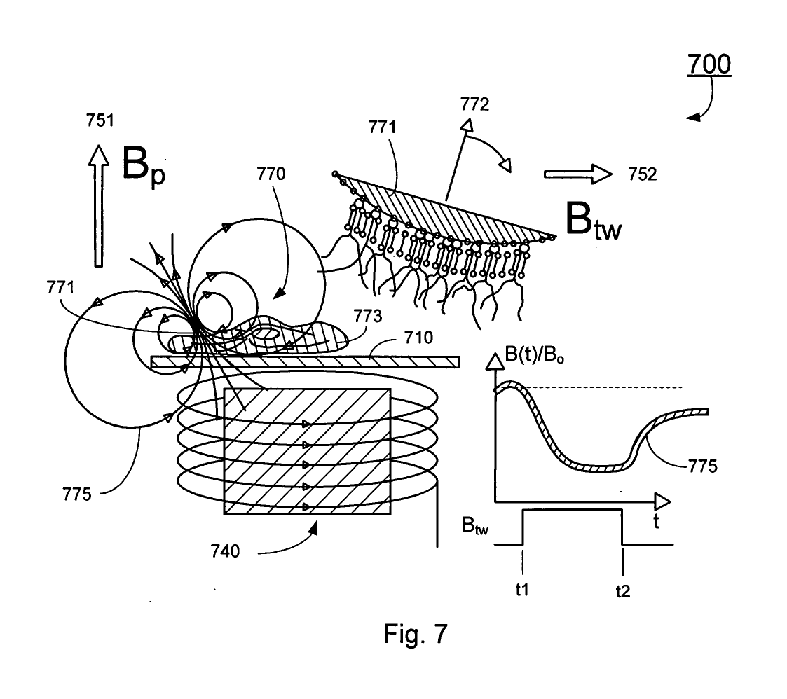

The ability to track the magnetic moment of magnetic nanoparticles and the ability to trap cells in the sensing volume of the SQUID microscope offered by practicing the present invention may find many applications in a wide spectrum of fields, including probing the mechanical properties and cell motility of a single cell, and the viscoelastic properties of the cytoskeleton of a single cell. Referring to FIG. 7, a method 700 of probing the mechanical properties and cell motility of single cells is schematically shown according to one embodiment of the present invention. The method 700 includes the following steps: at first a cell sample 770 containing cells 773 and magnetic beads 771 is provided, where each magnetic bead 771 is attached to a corresponding cell 773 to form a cell-bead unit such that when the magnetic bead 771 moves and/or rotates, the corresponding cell 773 moves and/or rotates accordingly. A microfluidic structure 710 defining a detecting zone capable of trapping a single cell 773 therein is also provided. Then a vector microscope 740 is positioned proximately to the detecting zone of the microfluidic structure, where the vector microscope has three SQUID sensors orthogonally oriented for simultaneously measuring three orthogonal components of a magnetic field of a cell trapped in the detecting zone. Next, the cell sample 770 is introduced into the microfluidic structure 710, where the cell sample 770 is regulated such that each cell-bead unit passes singly through the detecting zone. Then a first magnetic field BP is applied to the cell sample 770 along a first direction 751, where the first magnetic field BP has a short but strong magnetic pulse adapted for saturating magnetic moments 772 of the magnetic beads 771. After the magnetic moments of the magnetic beads are saturated, a second magnetic field B_{tw} is applied to the cell sample 770 along a second direction 752 orthogonally to the first direction 751, starting from time t1. The second magnetic field B_{tw} has a weak uniform magnetic field adapted for creating a torque on the magnetic beads 771 so as to cause them to rotate from a first orientation to a second orientation. The second magnetic field is also referred to a twisting field. Then, the second magnetic field B_{tw} is turned off at time t2, so as to allow the magnetic beads 771 to recover from the second orientation to the first orientation. A transient magnetic field 775 of the cell-bead unit in the detecting zone is continuously measured for a time period at least including time t1 and time t2. The measured magnetic field is related to the angular rotation of the magnetic bead of the cell-bead unit and therefore to the angular rotation of the corresponding cell. The angular rotation of the corresponding cell varies with and is a function of time and the strength of the second field, and is related to the mechanical properties and cell motility of the corresponding cell.

These and other aspects of the present invention are further described below.

### EXAMPLES AND IMPLEMENTATIONS OF THE INVENTION

Without intent to limit the scope of the invention, exemplary apparatus and methods and their related results according to the embodiments of the present invention are given below. Note again that titles or subtitles may be used in the examples for convenience of a reader, which in no way should limit the scope of the invention. Moreover, certain theories are proposed and disclosed herein; however, in no way they, whether they are right or wrong, should limit the scope of the invention.

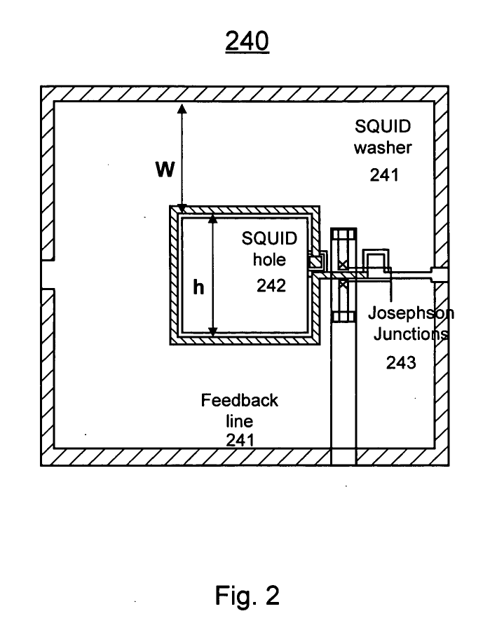

Referring to FIG. 2, a SQUID sensor 240 utilized in the flow cytometer shown in FIG. 1 is shown. In the embodiment, the SQUID sensor 240 is a directly-coupled low-temperature niobium based SQUID sensor. The SQUID sensor 240 is sized in about 150×150 μm . The SQUID sensor 240 includes a washer sized about w=30 μm defining a hole 242 sized about h=30 μm at the center of the SQUID sensor 240. The SQUID sensor 240 includes two Josephson junctions 243 and a feedback line 242. In one embodiment, the SQUID sensor 240 is characterized with a SQUID inductance, L, a Josephson junction (JJ) critical current, I_{c}, a JJ self-capacitance, C, and a shunt resistance, Rₙ. The design and the characteristics of the monolithic thin film SQUID sensors are described in Ref. [8]. Several key parameters of the SQUID sensor 240 are summarized in Table 1. As shown in FIG. 1, The SQUID sensor 140 is located in the vacuum space 165 of the dewar 160, and separated by a sapphire window 130 having a thickness g=25 μm from a microfluidic structure 110 having a channel 120 that is configured to transport a stream of fluid with magnetic particles 170 at a room temperature, whereby the SQUID sensor 140 defines a detecting (sensing) zone 150 in the microfluidic structure 110, which is under the SQUID sensor 140. In this embodiment, the sensor-to-particle distance, d, is about 100 μm. In the exemplary embodiment as shown in FIG. 1, the microfluidic structure 110 is adhered to the 25 μm thick sapphire vacuum window 130, which in turn, is mechanically clamped to the tail portion 162 of the dewar 160. A serpentine channel geometry 122 is chosen to provide a periodic mechanical modulation of a magnetic particle as it travels along the serpentine path of the channel 120. The cross-sectional dimension of the channel 120 has a width about w1=25 μm and a height about h1=15 μm which are adapted for accommodating a single magnetic particle 170. Other dimensions of the channel can also be utilized to practice the current invention. The microfluidic structure 110 is fabricated using poly(dimethylsiloxane) (PDMS) and replication molding [11-13].

TABLE 1
Characteristics of a SQUID sensor used for detection of
single magnetic particles. A_{eff} is the effective sensing
area, S_{φ}^{1/2}, S_{B}^{1/2} and Sₘ^{1/2} are the magnetic flux
noise, field resolution and moment sensitivity per
unit bandwidth at 1 Hz, respectively.

| A_{eff} | S_{φ}^{1/2} (1 Hz) | S_{B}^{1/2} (1 Hz) | Sₘ^{1/2} (1 Hz)@100 μm |
|---|---|---|---|
| (mm²) | (μφ₀/Hz^{1/2}) | (pT/Hz^{1/2}) | (Am²/Hz^{1/2}) |
| 6.64 × 10⁻³ | 4.0 | 1.8 | 9.0 × 10⁻¹⁸ |

Magnetic beads (SPHERO-CFM-60-5, Spherotec, Inc) are used as magnetic particles. These beads are made of chromium dioxide (CrO₂) uniformly coated with polystyrene forming micron sized particles. Each of the particles is formed with a size between about 6 and 8 μm in diameter (CFM-60-5), which has a CrO₂ content of 20% of the total bead volume [14]. The original concentration is diluted to a 0.005% weight to volume ratio using distilled water to form a bead suspension (a stream of fluid with magnetic particles). The bead suspension is placed in an ultra-sonic bath for several minutes to disperse aggregates. Prior to the injection of the bead suspension into the channel 120, the bead suspension is magnetized using an impulse magnetizer (IM-10-30 ASC Scientific) with a pulse amplitude of about 545 mT. The bead suspension is injected into the microfluidic channel 120 using a static pressure generated by compressed N₂ gas. The N₂ gas pressure is varied in the range from about 6.9 KPa to about 34.5 KPa to adjust the fluid velocity. Other types of pressurizers can also be utilized to practice the present invention. The bead suspension is regulated such that only a single bead traveles along the channel 120 through the detecting zone 150. A video camera on an inverted microscope is used to observe the beads flowing in the serpentine channel 120.

Once the bead suspension is introduced into the channel 120 of the microfluidic structure 110, the time trace of the magnetic field component perpendicular to the plane of the serpentine channel 120, which is the magnetic signatures of a single bead traveling through the detecting zone 150 of the serpentine channel 120, is recorded. In this example, the magnetic signatures of a single bead traveling through the detecting zone 150 of the serpentine channel 120 are recorded with an average signal-to-noise ratio of about 10:1.

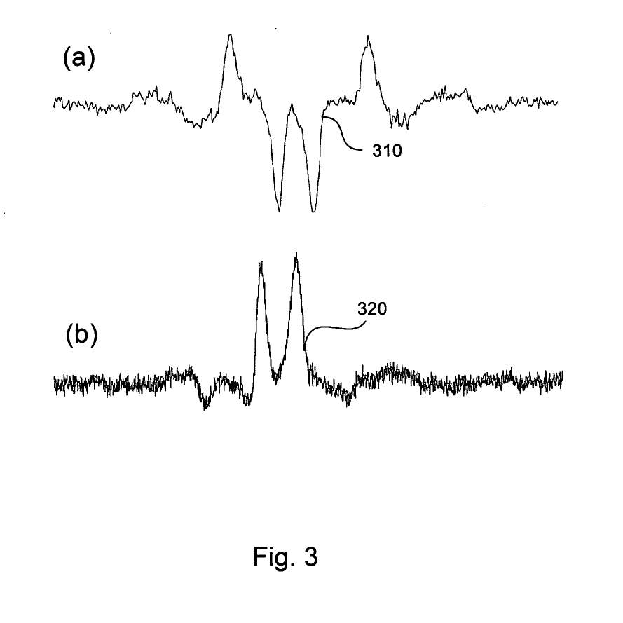

FIGS. 3a and 3b show two such magnetic signatures observed at a N₂ gas pressure of about 27.6 KPa at a sensor bandwidth of about 500 Hz and 2.5 KHz, respectively. The time traces 310 and 320 are significantly different in shape but have common features. This could be inferred from a comparable time separation between local extrema. The time traces 310 and 320 are fairly symmetrical around a well defined point in time. The symmetry resulted from geometrical constraints and the assumption that the magnetic beads maintain the orientation of their magnetic moments or cyclically change their moment along the serpentine channels. The geometrical constrains are the symmetrical channel layout and the position of the SQUID sensor 140 in the center 150 between two straight segments of the channel 120, as shown in FIG. 1a. The amplitude of the magnetic signatures varied in a range of less than one order of magnitude. To further investigate the characteristics of the signatures, a numerical model is devised to reproduce the time traces recorded by the SQUID sensor so as to recover the magnetic moment as the bead travels through the channel.

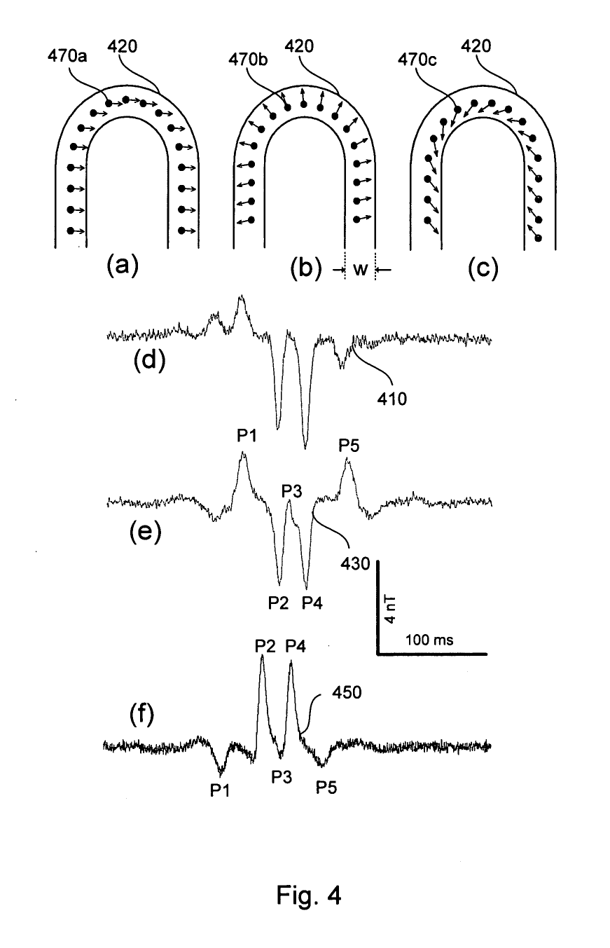

In the numerical model a single bead is represented as a magnetic dipole moving along a parameterized path on the x-y plane of the microfluidic channel reproducing the serpentine geometry. The simulated sensor predicted the z-component of the magnetic field integrated over the area of the sensor. Two different schemes for the movement of the bead inside the channel are investigated. In one embodiment, the bead 470a only experienced translational movement and the direction of the simulated dipole in space is kept constant along the trajectory 420, as shown in FIG. 4a. In the other embodiment shown in FIGS. 4b and 4c, in addition to the translational movement, the bead 470b (470c) experienced rotation in the x-y plane while traveling the curved segments of the path 420. The fluid velocity is larger on the inner radius compared to the outer radius of the turns in the serpentine, which could lead to a rotation of the particle. FIGS. 4b and 4c show the change in the direction of the magnetic moment of the particle 470b and 470c through the curved segments of the path 420 for each case.

As the particle 470a (470b or 470c) flows through the channel 420 of the model, for each time instant the magnetic flux at the position of the SQUID sensor by means of a 64-point two-dimensional Gaussian quadrature integration algorithm is evaluated over the effective area of the sensor. In one embodiment, the model is based on eight parameters: time offset, bead speed, x and y coordinates of the SQUID sensor, lift-off distance, and x, y and z components of the magnetic moment of the bead. The model parameters are obtained using the Nelder-Mead nonlinear optimization method to minimize the least squares difference between simulated and experimental signals. The noise on the recordings is represented in the model as white noise within the bandwidth set by the SQUID electronics.

FIG. 4d shows the simulated time trace 410 based on the assumption of a constant dipole orientation, corresponding to the embodiment of FIG. 4a. FIG. 4e shows the time trace 430 for the case where the moment changes direction according to the turns of the serpentine channels 420 shown in FIG. 4b, where the angle of the dipole in relation to the tangent of the trajectory is kept constant. Comparing these simulations to the experimental trace of FIG. 3a, it is clearly shown that the orientation of the magnetic moment did not stay constant. Another example of modeling a magnetic signature 450 of a single particle 470c traveling through the serpentine channel 420 is shown in FIG. 4f, which is obtained based on the measured time trace depicted in FIG. 3b. FIG. 4c shows the projection of the magnetic moment onto the x-y plane of the particle along the trajectory. These data also supports a cyclic change in the direction of the orientation to be a better description of the behavior of the particle inside the microfluidic channel.

The data modeling allows one also to recover all three components of the magnetic moment which gives a total magnetic moment of 4.7×10⁻¹⁴ Am² and 2.8×10⁻¹⁴ Am² for the embodiments of FIGS. 3a and 3b, respectively. The spread in magnetic moment could be caused by field inhomogeneities during the pulse magnetization and non uniform particle characteristics. The fitting procedure is very consistent for predicting the speed of the particle and the lift-off or the distance between the sensor and the plane of the trajectory. The particle speed observed in the embodiments of FIGS. 3a and 3b is of about 16.3 mm/s and 15.9 mm/s, respectively. The lift-off is about 95±3 μm. Such small variation could be expected for a 6 μm bead moving in a channel having a height about 15 μm.

The performance of the flow cytometer of the present invention is further improved by optimizing the low-temperature-superconductivity (LTS) SQUID design and reducing the spacing between the microfluidic channels and the SQUID sensor using a microfabricated SiN window to less than 50 μm. For example, the detection sensitivity to magnetic moment of a magnetic particle is increased by much more than one order of magnitude for reduced spacings between the microfluidic channels and the SQUID sensor. The dramatic increase of the field sensitivity results from the magnetic field decreasing as 1/r³ with distance. The increased sensitivity would allow one to improve the ability to discriminate and to increase the bandwidth for higher throughput operation by at least a factor of 100. This increases the current signal to noise ratio by at least a factor of 100 and reduce the error rate, thereby, increasing the discriminatory power for high content sorting operations.

Optimizing the performance of a washer-type SQUID design: To obtain a high spatial resolution of a flow cytometer of the present invention, a dc-SQUID sensor is adapted to directly detect the sample's magnetic field. FIG. 2 shows one embodiment of a SQUID sensor 240 chip layout with integrated feedback line 241 according to the present invention. The dimensions of the sensor are 120 by 120 μm, where w and h (washer and hole dimensions) are 40 μm respectively. Two resistively shunted Josephson junctions (JJ) 243 are fabricated by Hypres using a Nb/Al trilayer process with Mo thin film shunt resistors. The photolithographic process imposes a minimum JJ diameter of 3.5 μm, which results in a self-capacitance, C, of about 0.4 pF/JJ, and a critical current, I_{c}, of about 25 μA at a process-specific critical current density of about 100 A/cm². Table 2 shows the characteristic parameters of the bare SQUID sensor design, where A_{eff} is an effective sensing area of the SQUID sensor 240, A_{geo}=(w+h)² is an geometrical area of the SQUID sensor 240, L is a SQUID inductance, I_{c} is a critical current, Rₙ is a resistance of the shunt, ΔV is a peak-to-peak modulation depth, β_{L}=2LI_{c}/φ₀, S₁₀₀(f)^{1/2}ₜₕₑₒ is a theoretical magnetic flux noise, S₁₀₀(f)^{1/2} and S_{B}(f)^{1/2} are the measured magnetic flux and field noise per unit bandwidth at the specified frequency, respectively.

TABLE 2
Characteristics of a SQUID sensor used for detection of a single magnetic particle.

| h | A_{eff} | A_{geo} | L | I_{c} | R_{α} | ΔV | | S_{φ}^{1/2}ₜₕₑₒ | S_{φ}^{1/2} | S_{B}^{1/2} | S_{B}^{1/2} |
|---|---|---|---|---|---|---|---|---|---|---|---|
| (μm) | (Mm²) | (mm²) | (pH) | (μA) | (Ω) | (μV) | β_{L} | (μφₒ/Hz^{1/2}) | (μφₒ/Hz^{1/2}) | (pT/Hz^{1/2}) | (pT/Hz^{1/2}) |
| 40 | 7.4 × 10⁻³ | 6.4 × 10⁻³ | 58.1 | 21.81 | 2.6 | 16 | 1.22 | 3.06 | 5 (1 kHz) | 3.5 (1 Hz) | 1.4 (1 kHz) |

The optimum sensitivity for such a dc-SQUID sensor 240 operated at 4.2 K requires that the JJ parameters and the SQUID inductance, L, are chosen to satisfy the two constraints, β_{c}=2πI_{c}Rₙ²C/φ₀≦0.7 and β_{L}=2LI_{c}/φ₀≅1, where φ₀ is a flux quantum of about 2×10⁻¹⁵ Wb is, I_{c} is the JJ critical current, and Rₙ is the shunt resistance. The optimization for such a bare SQUID sensor 240 is straightforward and requires reducing the operation temperature T of the SQUID sensor, the SQUID inductance L and the JJ self-capacitance C. Table 3 lists the characteristics of different SQUID sensors optimized for various spatial resolutions.

TABLE 3
Characteristics of two SQUID sensors used for
detection of a single magnetic particle.

| Hole | washer | JJ - size | Rₙ | | | S_{φ}^{1/2} Theo | S_{B}^{1/2} Theo |
|---|---|---|---|---|---|---|---|
| (μm) | (μm) | (μm) | (Ω) | β_{C} | β_{L} | (μφₒ/Hz^{1/2}) | (pT/Hz^{1/2}) |
| 30 | 5 | 4.0 | 4.5 | 0.63 | 1.04 | 0.42 | 0.71 |
| 60 | 5 | 2.5 | 12.0 | 0.68 | 0.96 | 0.63 | 0.31 |

By adjusting the Josephson junction size and the normal state resistance Rₙ of a SQUID sensor, the performance of the SQUID sensor can substantially be improved. Therefore, when fabricating the SQUID sensor chips, Rₙ and the Josephson junction size are varied around the optimum values in order to obtain the best performance.

Reducing the thickeness of the vacuum window: The reasons for this are twofold: (i) the 1/r³ falloff of the magnetic field translates into greatly improved field sensitivity from a smaller spacing, and (ii) the spatial resolution of the instrument (in terms of resolving magnetization) is limited by the sensor-to-sample spacing when the spacing is larger than the sensor size. The main limitations on reducing the spacing are the thickness g of the vacuum window, which is in a range of about 25-50 μm in one embodiment. This is further reduced by back etching silicon nitride coated wafers using micromachining techniques like photolithography and wet etching. Using the techniques, a silicon nitride window is made much thinner than that from sapphire because of its large elastic modulus. For such a silicon nitride window, the window thickness g is reduced to about to 5-10 μm.

Implementation of active pneumatic valves to deflect and sort magnetically labeled analytes: In one embodiment, a flow cytometer of the present invention integrates a SQUID sensor with SQUID-triggered pneumatic valves for sorting of magnetic labels. The pneumatic valves are formed with at least two elastomer layers. One layer contains channels for flowing liquids (flow layer), and the other layer contains channels that when pressurized with air or nitrogen serve as valves and pumps for the flow channels (control layer). The active pneumatic valves are fabricated in the VIIBRE (Vanderbilt Institute of Integrative Research and Education) Class-100 clean room using soft lithography. Molds are made by spin-coating photoresist onto silicon wafers, exposing the coated wafer with light transmitted through a patterned photomask, then rinsing away the unexposed photoresist. Developed wafers form master molds for repeated casting of a given structure. PDMS is cast onto these masters either by spin coating a thin layer or by pour casting a thick layer into a reservoir containing the master. To form multilayer devices, a technology pioneered by Stephen Quake et al. is adapted to fabricate the PDMS multi-layer chips with a fluidic and a control layer separated by a thin membrane [15, 16]. Intersections of fluidic and control layers form valves that are actuatable by pressurizing the control line. Inline valves placed in series serve as peristaltic pumps. FIG. 5 shows a typical intersection between a control line 514 and a fluidics line 513 that form a valve 516 to control fluid flow. Computer control of an external pneumatic valve manifold allows one to program repetitive sequences of open-close cycles for operating a series of fluid valves as peristaltic pumps [15]. In one embodiment, a T-junction with two pneumatic valves is used to discriminate magnetically labeled cells. The SQUID sensor signal is used to trigger the valves via control software and thus sort each cell into its designated bin (port). In one embodiment, a small permanent magnet and/or a magnetic coating in close proximity to the microfluidic channel upstream from the SQUID sensing region is utilized to polarize the beads so that the SQUID sensor maximally detects the magnetic signatures from each bead. As shown below, a vector-SQUID microscope enables additional discrimination that may eliminate the need for bead polarization and so allow more complex bead manipulations in the microfluidic channels.

Selection and purchase of magnetic bead assortment: A number of sets of magnetic beads are obtained from Sphero-tec Inc. (for example SPHERO CFM 60-5) with controlled amounts of magnetic nanocrystals embedded in the polystyrene core. All these magnetic beads have magnetic moments spanning the detection range of the SQUID sensor. Furthermore, each of the magnetic beads is magnetize at different field strength so as to obtain a desired amount of remnant magnetization. In one embodiment, more than 100 distinguishable magnetic moments are achieved. By running the bead solution through a magnetic sorter, one could further pre-select beads that display distinct magnetic moments within tolerances that approach the limit of the detection resolution, and reject beads with magnetic moments that are aberrantly high, low or “in-between” bins.

The magnetic beads may be manufactured with optical labels such as quantum dots and magnetic nanoparticles, creating a truly 2-dimensional analyte identification matrix.

Cell sorting using magnetic markers: In one embodiment, the cell sorting is preformed with an artificially created mixture of two cell types: the CD51 positive (CD51+) melanoma cell (m21) and the CD51 negative (CD51−) melanoma cell (m21-L). To facilitate downstream detection of the cell-bead agglomeration and assess the sorting success, each of the m21 cells is labeled with red cell-tracker dye, and each of the m21-L cells is labeled with green cell-tracker dye at first. The cell-tracker dye remains on the cells for the duration of the cell sorting. The mixture of the m21 and m21-L cells is then be incubated with anti-CD51 antibody (Ab1) followed by magnetic beads coated with a secondary antibody (Ab2). Ab2 is specific to Ab1, and the magnetic beads therefore only bind to the m21 cells and free Ab1. Free magnetic beads do not interfere with downstream measurements, but they may be partially removed from the cell mixture using bulk magnetic separation techniques since their mobility is higher than the cell-bound beads. The final mixture of cells is introduced into the microfluidic structure positioned under the SQUID sensor and sorted according to the presence or absence of a magnetic moment. The magnetically-labeled cells may be further purified by magnetic bulk separation. The sorting efficacy will then be determined by the flow cytometer of the two sorted populations. The number of red and green fluorescent cells in each population is quantified and compared with populations sorted with conventional flow cytometer. Due to the extremely sensitive magnetic detection capability of the SQUID sensor, the first-pass separation of the m21 and m21-L cells is more efficient, as compared with that of the conventional flow cytometer. Additionally, background fluorescence is substantially reduced in the magnetically-labeled cell sample due to bulk magnetic separation/purification prior to the SQUID flow cytometer analysis according to one embodiment of the present invention.

This simple bulk separation method combined with highly a discriminate sorting apparatus may greatly improve the overall sensitivity of a bead-based assay by eliminating the vast majority of unwanted contaminates prior to microfluidic detection and sorting. Magnetic-bead based assays and sorting are further benefited by the ability to impose force at a distance on the beads while in the incubation solution using a permanent magnet.

Selective protein detection using antibody-coated magnetic beads: In one embodiment, the magnetic bead-based, multiplexed detection of proteins is shown by identification and molecular binding of seven cytokines from a standard mixture of Human Th1/Th2 cytokines (BD-Pharmingen, San Diego, Calif.) using streptavidin-biotin magnetic bead-antibody complexes and secondary fluorescent antibodies in a sandwich immunoassay [17]. After incubation with a solution containing the cytokine standards and fluorescent secondary antibodies, the magnetic beads are sorted with the invented SQUID microfluidics system into seven pools. The resulting pools are purified of free antibodies by iterative bulk magnetic separation and analyzed by flow cytometer. The Becton-Dickinson Human Th1/Th2 Cytokine Cytometric Bead Array (non-magnetic) is used as a benchmark for the experiment. The sensitivity, and therefore the potential content-capacity, of the invented SQUID microfluidics system is demonstrated for multiplexed protein assays. It is estimated the invented SQUID microfluidics system has the capability of discriminating the magnetic moments of over 1000 unique magnetic beads. This capability would pave the way for a similarly large number of simultaneous detection/capture assays in a small reaction volume, with analyte recovery and quantitation facilitated by the ultra-sensitive SQUID and microfluidics sorting. According to the embodiment of the present invention, seven human Th1/Th2 cytokines in solution including interleukin (IL)-2, IL-4, IL-5, IL-6, IL-10, tumor necrosis factor (TNF) and interferon-γ IFN-γ are simultaneously detected. The relative cytokine concentration in solution by fluorescence intensity measurement via flow cytometer is estimated. Furthermore, it is demonstrated that the background noise with associated increased sensitivity in the flow cytometer phase is reduced due to bulk magnetic separation/purification of bead-cyotokine complexes.

A vector SQUID microscope: Referring to FIG. 6, a vector SQUID microscope 600 capable of measuring instantaneously all three vector components of the magnetic field of a particle directly without the need to force the particle through a serpentine channel is shown according to one embodiment of the present invention. Detecting the vector nature not only allows one to shorten the detection path and improve the ability to discriminate the magnetic moment of nanoparticles (cells), but also allows one to probe the elastic properties and the force generation on the single cell level. As shown in FIG. 6, the vector SQUID microscope 600 includes three orthogonally oriented monolithic SQUID sensors chips 630a, 630b and 630c. These SQUID sensors 630a, 630b and 630c are placed at the corners (tip) 642 of a sapphire cube 640, with the cube diagonally aligned normal to the scanning plane in operation. The cube 640 is machined from a solid sapphire rod, which gets inserted into standard collets at the very tip of a cold finger assembly. This geometrical orientation of the SQUID sensors 630a, 630b and 630c enables simultaneous, equidistant measurements of all three components of the field of a magnetic particle. In practice, the contact pad layout may be modified to allow for connections to the sensor 630a, 630b and 630c while mounted on the cube structure (tip) 642 of the sapphire rod 640. One of the main advantages of this configuration is that it allows one to achieve high spatial resolution with equally spaced sensors 630a, 630b and 630c very close to the sample. The electrical connections on the cube structure (tip) 642 of the sapphire rod 640 to the SQUID sensors 630a, 630b and 630c are made using metal films 645 evaporated through photoresist masks. FIG. 6a shows a sensor measuring vertical component of a magnetic field, which includes a sapphire rod 620 wound with Nb—Ti pickup coil 610 with a diameter about 250 μm and height about 150 μm. FIG. 6b shows schematically a vector sensor 600 includes three, planar SQUID-bearing plates 631a, 631b and 631c orthogonally mounted on the sapphire tip 640.

According to the present invention, microfluidics and SQUID microscopy can be used to probe the mechanical properties and cell motility of single cells. The approach is based on the ability to track the magnetic moment of magnetic nanoparticles and the ability to trap cells in the sensing volume of the SQUID microscopy. A method of magnetic twisting cytometer to study the motility and rheological properties of pulmonary macrophages has been reported [18].

Magnetic twisting cytometer: The magnetic twisting cytometer is gaining wide applicability as a tool for the investigation of the rheological properties of cells and the mechanical properties of receptor-cytoskeletal interactions [19-21]. This methodology uses small magnetic nanoparticles, embedded within the cell (either through phagocytosis or injection), or bound to the cell membrane by coupling of ligand-coated beads of specific cell membrane receptors [19, 20, 22]. FIG. 7 shows schematically a process 700 of probing the mechanical properties and cell motility of single cells. At first, a cell sample 770 containing cells 773 and magnetic beads 771 is prepared, where each magnetic bead 771 is attached to a corresponding cell 773 to form a cell-bead unit such that when the magnetic bead 771 moves and/or rotates, the corresponding cell 773 moves and/or rotates accordingly. A microfluidic structure 710 defining a detecting zone capable of trapping a single cell 773 therein is also provided. Then a vector microscope 740 is in communication with the detecting zone of the microfluidic structure 710 for simultaneously measuring three orthogonal components of a magnetic field of a cell 773 trapped in the detecting zone. Then the magnetic bead 771 is magnetized by a short but strong magnetic pulse, Bₚ, along a first direction (magnetization direction) 751. The short but strong magnetic pulse Bₚ is adapted to saturate the magnetic moment of the magnetic bead 771, so that the magnetic moment of the magnetic bead 771 is orientated at a firs orientation. Second, a weak uniform “twisting” field, B_{tw}, is applied from time t1 to time t2, along to a second direction 751 that is orthogonally to the magnetization direction 751. This creates a torque on the magnetic bead 771 which causes it to rotate from the first orientation to a second orientation. Then, the twisting field B_{tw} is turned off at time t2, and the cell-bead unit (preparation) is allowed to recover from the second orientation to the first orientation.

The magnitude of the applied specific torque (torque per unit volume) and resulting angular rotation of the beads is measured. The relationships between torque and angular rotation obtained as a function of time and twisting field strength are the primary data used to obtain rheological properties specific parameters such as an apparent elastic modulus (or stiffness) and a viscosity. The necessary calibration constants are obtained from measurements of beads in a viscous standard if the beads are internalized [19]. It is important to realize that all published measurements are made on populations of cells which themselves may exhibit heterogeneous rheological properties. Furthermore, the interpretation of the angle of the assembly of cells relative to the twisting field is problematic if intrinsic relaxation is present.

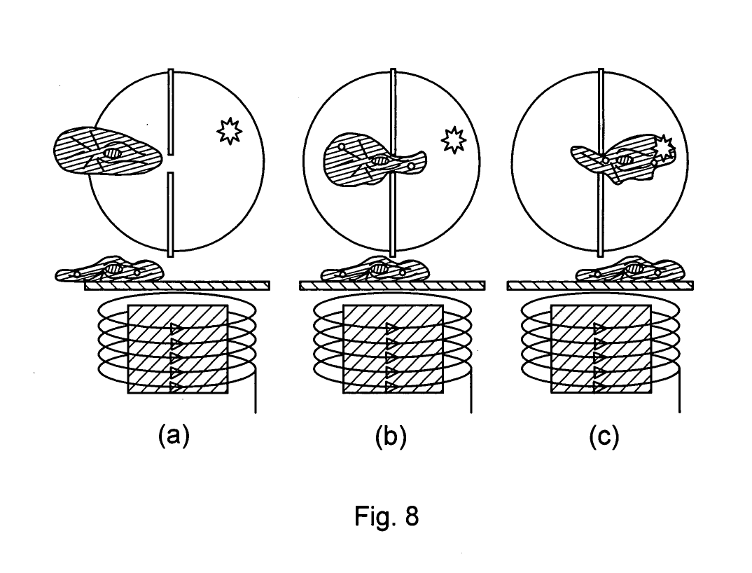

According to one embodiment of the present invention, cell magnetic twisting cytometer on single cells during functional changes is performed, as shown in FIG. 7. In one embodiment, FIG. 8 shows schematically morphology changes of a tumor cell cytoskeleton during extravasation, which is an important process in tumor metastasis. The ability to measure the vector components of the magnetic field of the tumor cell-bead unit allows one to determine the position and the rotational state of the bead, and thus the mechanical properties of the tumor cell. Furthermore, it allows one to asses the anisotropies of the stiffness, a parameter that is difficult to measure in non adhered cells.

As a comparison, an established protocol is used to magnetically induce force applied directly to the cytoskeleton through integrin-coupled magnetic beads coated with Arg-Gly-Asp (RGD) peptide [23] and measure the orientation of the magnetic bead to probe the stiffness and the viscosity of single cells. The stiffness of the cytoskeleton can be varied by incubation of the cardiac myocytes in a dilute taxol solution which is known to cause microtubule hyperpolymerization and causes an increase in cell stiffness and apparent viscosity [24]. The present invention, among other things, may lead to a better understanding of (i) how migrating cells prioritize and process directional, environmental cues; (ii) how these genetically-coded and environmentally-regulated processes translate the random-walk motility of cells in vitro to directed cell migration; and (iii) how directed cell migration potentiates angiogenesis and extravasation in the tumor.

The present invention, among other things, discloses low-temperature-superconductivity (LTS) SQUID microscopy in combination with a flow path modulation and/or one or more pneumatic valves in a microfluidic device provided the sensitivity to detect and identify the magnetic moment of a single magnetic particle in flight with a magnetic moment as small as 10⁻¹⁸ Am²/Hz^{1/2}. The invented systems and methods significantly enhance the sensitivity of magnetic field detection, allowing the discrimination of tagged cells according to the magnetic moment of the label for high content and high through put applications. The present invention may find many applications in a wide spectrum of fields, including probing the mechanical properties and cell motility of a single cell, and the viscoelastic properties of the cytoskeleton of a single cell.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments are chosen and described in order to explain the principles of the invention and their practical application so as to activate others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

### LIST OF REFERENCES

[1]. Häfeli, U., Schütt, W., Teller, J., Zborowski, M., Scientific and Clinical Applications of Magnetic Carriers (New York, 1997).
[2]. I. Safarik, M. Safarikova, Journal of Chromatography B 722, 33 (1999).
[3]. J. C. Rife, M. M. Miller, P. E. Sheehan, C. R. Tamanaha, M. Tondra, L. J. Whitman, Sensors and Actuators A: Physical 107, 209 (2003).
[4]. H. A. Ferreira, D. L. Graham, P. P. Freitas, J. M. S. Cabral, J. Appl. Phys. 93, 7281 (2003).
[5]. P. A. Besse, G. Boero, M. Demierre, V. Pott, R. Popovic, Appl. Phys. Lett. 80, 4199 (2002).
[6]. D. R. Baselt, G. U. Lee, K. M. Hansen, L. A. Chrisey, R. J. Colton, Proceedings of the Ieee 85, 672 (1997).
[7]. M. M. Miller, G. A. Prinz, S. F. Cheng, S. Bounnak, Appl. Phys. Lett. 81, 2211 (2002).
[8]. F. Baudenbacher, L. E. Fong, J. R. Holzer, M. Radparvar, Appl. Phys. Lett. 82, 3487 (2003).
[9]. Holzer J. R., Fong L. E., Sidorov V. Y., Wikswo J. P., Baudenbacher F., Accepted to Biophysical Journal 87, 4326 (2004).
[10]. F. Baudenbacher, N. T. Peters, J. P. Wikswo, Jr., Rev. Sci. Instrum. 73, 1247 (2002).
[11]. D. C. Duffy, J. C. McDonald, O. J. A. Schueller, G. M. Whitesides, Analytical Chemistry 70, 4974 (1998).
[12]. Y. N. Xia, G. M. Whitesides, Annual Review of Materials Science 28, 153 (1998).
[13]. A. Y. Fu, C. Spence, A. Scherer, F. H. Arnold, S. R. Quake, Nature Biotechnology 17, 1109 (1999).
[14]. Spherotech Product Catalogue, 2004, Spherotech, Inc. Libertyville, Ill.
[15]. Unger, M A, Chou, H P, Thorsen, T, Scherer, A, Quake, S R. Monolithic Microfabricated Valves and Pumps by Multilayer Soft Lithography. Science, 288, 113-116, 2000.
[16]. Quake, S R and Scherer, A. From Micro-to Nanofabrication With Soft Materials. Science, 290, 1536-1540, 2000.
[17]. Morgan, E, Varro, R, Sepulveda, H, Ember, J A, Apgar, J, Wilson, J, Lowe, L, Chen, R, Shivraj, L, Agadir, A, Campos, R, Ernst, D, Gaur, A. Cytometric Bead Array: a Multiplexed Assay Platform With Applications in Various Areas of Biology. Clinical Immunology, 110, 252-266, 2004.
[18]. Valberg, P A and Albertini, D F. Cytoplasmic Motions, Rheology, and Structure Probed by A Novel Magnetic Particle Method. Journal of Cell Biology, 101, 130-140, 1985.
[19]. Valberg, P A and Butler, J P. Magnetic Particle Motions Within Living Cells—Physical Theory and Techniques. Biophysical Journal, 52, 537-550, 1987
[20]. Valberg, P A and Feldman, H A. Magnetic Particle Motions Within Living Cells—Measurement of Cytoplasmic Viscosity and Motile Activity. Biophysical Journal, 52, 551-561, 1987.
[21]. Valberg, P A and Albertini, D F. Cytoplasmic Motions, Rheology, and Structure Probed by a Novel Magnetic Particle Method. J.Cell Biol., 101, 130-140, 1985
[22]. Wang, N, Butler, J P, Ingber, D E. Mechanotransduction Across the Cell-Surface and Through the Cytoskeleton. Science, 260, 1124-1127, 1993.
[23]. Tagawa, H, Wang, N, Ingber, D E, Cooper, G. Increased Cytoskeletal Viscosity in Pressure-Overload Cardiac-Hypertrophy. Circulation, 92, 3154-3154, 1995.
[24]. Tagawa, H, Wang, N, Narishige, T, Ingber, D E, Zile, M R, Cooper, G. Cytoskeletal Mechanics in Pressure-Overload Cardiac Hypertrophy. Circulation Research, 80, 281-289, 1997.

What is claimed is:

1. A flow cytometer, comprising:
a. a microfluidic structure defining a channel with a periodically modulated path for transporting a stream of fluid with magnetic particles along the modulated path; and
b. a superconducting quantum interference device (SQUID) sensor positioned over the microfluidic structure to define a detecting zone in the microfluidic structure for detecting magnetic signatures of a magnetic particle passing along the periodically modulated path through the detecting zone,
wherein in use the stream of fluid with magnetic particles is regulated such that each magnetic particle passes singly along the periodically modulated path through the detecting zone.

2. The flow cytometer of claim 1, further comprising a dewar having a tail portion configured to house the SQUID sensor in relation to the microfluidic structure such that there is a distance, d, between the SQUID sensor and the stream of fluid with magnetic particles passing along the microfluidic device structure.

3. The flow cytometer of claim 2, further comprising a window member having a first surface and an opposite, second surface defining a thickness, g, therebetween and positioned between the tail portion of the dewar and the microfluidic device structure, wherein the thickness g is less than the distance d and in a range of from about 1 μm to about 50 μm.

4. The flow cytometer of claim 3, wherein the thickness g of the window member is preferably in a range of from about 5 μm to about 10 μm.

5. The flow cytometer of claim 1, further comprising an injecting member configured to introduce the stream of fluid with magnetic particles into the channel of the microfluidic structure.

6. The flow cytometer of claim 5, further comprising means for driving the stream of fluid with magnetic particles to flow along the channel of the microfluidic structure.

7. The flow cytometer of claim 6, wherein the driving means comprises a pressurizer in communication with the channel of the microfluidic structure capable of applying a predetermined amount of pressure thereto.

8. The flow cytometer of claim 1, further comprising a permanent magnet placed proximately to the channel of the microfluidic structure for polarizing each of the magnetic particles before it moves into the detecting zone.

9. The flow cytometer of claim 1, further comprising means for sorting each of the magnetic particles according to its detected magnetic signatures.

10. The flow cytometer of claim 1, wherein the channel of the microfluidic structure has a cross-sectional dimension sized to accommodate a single magnetic particle.

11. The flow cytometer of claim 10, wherein the microfluidic structure is made of poly(dimethylsiloxane) (PDMS).

12. The flow cytometer of claim 1, wherein the SQUID sensor comprises a directly-coupled low-temperature niobium based SQUID sensor.

13. The flow cytometer of claim 1, wherein the SQUID sensor comprises a washer-type SQUID sensor characterized with a SQUID inductance, L, a Josephson junction (JJ) critical current, I_{c}, a JJ self-capacitance, C, and a shunt resistance, Rₙ.

14. The flow cytometer of claim 14, wherein the SQUID sensor is adapted such that when the SQUID operates at a temperature of about 4.2 K, the SQUID inductance L, the JJ critical current I_{c}, the JJ self-capacitance C, and the shunt resistance Rₙ satisfy the relationships of β_{c}=2πI_{c}Rₙ²C/φ₀≦0.7 and β_{L}=2LI_{c}/φ₀≅1, wherein φ₀ is a flux quantum of about 2×10⁻¹⁵ Wb.

15. The flow cytometer of claim 1, wherein the stream of fluid with magnetic particles comprises a stream of biological analytes, each biological analyte hosting a magnetic bead having a unique magnetic moment.

16. The flow cytometer of claim 15, wherein the magnetic bead comprises an amount of magnetic nanoparticles embedded in the core of the bead and magnetized such that the magnetic bead has a desired amount of remnant magnetization.

17. The flow cytometer of claim 15, wherein the magnetic bead has an analyte-specific surface coating.

18. The flow cytometer of claim 15, wherein the-magnetic bead has an optical label including quantum dots.

19. The flow cytometer of claim 15, wherein the stream of biological analytes comprises one or more types of cells.

20. The flow cytometer of claim 19, wherein each cell is labeled with a cell-tracker dye.

21. The flow cytometer of claim 15, wherein the stream of biological analytes comprises one or more types of proteins.

22. The flow cytometer of claim 15, wherein the magnetic signatures of a magnetic particle comprises a temporal magnetic filed associated with the magnitude and the orientation of the magnetic moment of the magnetic particle passing through the detecting zone.

23. A flow cytometer, comprising:
a. a microfluidic structure having at least a first layer defining a fluidic channel, a second layer defining a control channel, and a membrane placed between the first layer and the second layer, wherein the fluidic channel and the control channel are aligned to form one or more intersections therebetween, each intersection defining a valve such that the fluidic channel and the control channel are in communication with each other through the one or more valves, wherein the fluidic channel is configured to transport a stream of fluid with magnetic particles, wherein the control channel is configured to individually actuate and/or de-actuate each of the one or more valves, wherein when one of the one or more valves is actuated, it allows a stream of fluid to flow from one side to the other side of the valve along the fluidic channel and vice versus, and wherein when one of the one or more valves is de-actuated, it allows no stream of fluid to flow from one side to the other side of the valve along the fluidic channel and vice versus; and
b. a superconducting quantum interference device (SQUID) sensor positioned over the microfluidic structure to define a detecting zone in the microfluidic structure for detecting magnetic signatures of a magnetic particle passing through the detecting zone.

24. The flow cytometer of claim 23, further comprising a dewar having a tail portion configured to house the SQUID sensor in relation to the microfluidic structure such that there is a distance, d, between the SQUID sensor and the stream of fluid with magnetic particles passing along the microfluidic device structure.

25. The flow cytometer of claim 24, further comprising a window member having a first surface and an opposite, second surface defining a thickness, g, therebetween and positioned between the tail portion of the dewar and the microfluidic device structure, wherein the thickness g is less than the distance d and in a range of from about 1 μm to about 50 μm.

26. The flow cytometer of claim 23, further comprising an injecting member configured to introduce the stream of fluid with magnetic particles into the channel of the microfluidic structure.

27. The flow cytometer of claim 26, further comprising means for driving the stream of fluid with magnetic particles to flow along the channel of the microfluidic structure.

28. The flow cytometer of claim 23, further comprising a permanent magnet placed proximately to the channel of the microfluidic structure for polarizing each of the magnetic particles before it moves into the detecting zone.

29. The flow cytometer of claim 23, further comprising means for sorting each of the magnetic particles according to its detected magnetic signatures.

30. The flow cytometer of claim 29, wherein the sorting means comprises a controller in communication with the SQUID sensor and the one or more valves for receiving the detected magnetic signatures of each of the magnetic particles and generating a corresponding trigger signal for each of the magnetic particles to actuate and/or de-actuate each of the one or more valves, thereby sorting each magnetic particle into its designated port.

31. The flow cytometer of claim 23, wherein the channel of the microfluidic structure has a cross-sectional dimension sized to accommodate a single magnetic particle.

32. The flow cytometer of claim 31, wherein the channel of the microfluidic structure is formed with a periodically modulated path.

33. The flow cytometer of claim 31, wherein the channel of the microfluidic structure is formed with a T-shape junction.

34. The flow cytometer of claim 23, wherein the SQUID sensor comprises a directly-coupled low-temperature niobium based SQUID sensor.

35. The flow cytometer of claim 23, wherein the SQUID sensor comprises a washer-type SQUID sensor.

36. The flow cytometer of claim 23, wherein the stream of fluid with magnetic particles comprises a stream of biological analytes, each biological analyte hosting a magnetic bead such that each of the magnetic particles has a unique magnetic moment.

37. A flow cytometer, comprising:
a. a microfluidic structure having a channel and one or more valves formed on the channel, wherein the fluidic channel is configured to transport a stream of fluid with magnetic particles, wherein when one of the one or more valves is actuated, it allows a stream of fluid to flow from one side to the other side of the valve along the fluidic channel and vice versus, and wherein when one of the one or more valves is de-actuated, it allows no stream of fluid to flow from one side to the other side of the valve along the fluidic channel and vice versus; and
b. a superconducting quantum interference device (SQUID) sensor positioned over the microfluidic structure to define a detecting zone in the microfluidic structure for detecting magnetic signatures of a magnetic particle passing through the detecting zone.

38. The flow cytometer of claim 37, further means for sorting each of the magnetic particles according to its detected magnetic signatures.

39. The flow cytometer of claim 38, wherein the sorting means comprises a controller in communication with the SQUID sensor and the one or more valves for receiving the detected magnetic signatures of each of the magnetic particles and generating a corresponding trigger signal for each of the magnetic particles to actuate and/or de-actuate each of the one or more valves, thereby sorting each magnetic particle into its designated port.

40. A method of detecting magnetic particles, comprising the steps of:
a. providing a microfluidic structure having a fluidic channel and a detecting zone defined with the fluidic channel;
b. introducing a stream of fluid with magnetic particles into the fluidic channel;
c. driving the stream of fluid with magnetic particles to flow along the fluidic channel, wherein the stream of fluid with magnetic particles is regulated such that each magnetic particle passes singly through the detecting zone; and
d. detecting magnetic signatures of a magnetic particle passing through the detecting zone.

41. The method of claim 40, wherein the detecting step is performed with a superconducting quantum interference device (SQUID) sensor that is positioned over the detecting zone such that there is a distance, d, between the SQUID sensor and the stream of fluid with magnetic particles passing through the detecting zone.

42. The method of claim 40, further comprising the step of sorting each of the magnetic particles according to its detected magnetic signatures.

43. The method of claim 42, wherein the fluidic channel of the microfluidic structure has a periodically modulated path.

44. The method of claim 42, wherein the fluidic channel of the microfluidic structure has a T-shape junction.

45. The method of claim 42, wherein the fluidic channel of the microfluidic structure has one or more valves, wherein when one of the one or more valves is actuated, it allows a stream of fluid to flow from one side to the other side of the valve along the fluidic channel and vice versus, and wherein when one of the one or more valves is de-actuated, it allows no stream of fluid to flow from one side to the other side of the valve along the fluidic channel and vice versus.

46. The method of claim 45, wherein the sorting step comprises the steps of:
a. receiving the detected magnetic signatures of each of the magnetic particles; and
b. generating a corresponding trigger signal for each of the magnetic particles to actuate and/or de-actuate each of the one or more valves, thereby sorting each magnetic particle into its designated port.

47. The method of claim 40, wherein the stream of fluid with magnetic particles comprises a stream of biological analytes, each biological analyte hosting a magnetic bead such that each of the magnetic particles has a unique magnetic moment.

48. A method of discriminating and/or sorting biological analytes, comprising the steps of:
a. preparing a magnetically-labeled analyte sample;
b. providing a flow cytometer comprising:
(i) a microfluidic structure defining a channel; and
(ii) a superconducting quantum interference device (SQUID) sensor positioned over the microfluidic structure to define a detecting zone in the microfluidic structure for detecting magnetic signatures of a magnetic particle passing along the channel through the detecting zone;
c. introducing the magnetically-labeled analyte sample into the channel of the microfluidic structure; and
d. detecting magnetic signatures of each analyte of the magnetically-labeled analyte sample passing along the channel through the detecting zone so as to sort the magnetically-labeled analyte sample according to its detected magnetic signatures.

49. The method of claim 48, wherein the magnetically-labeled analyte sample comprises CD51 positive (CD51+) melanoma cells (m21) and CD51 negative (CD51−) melanoma cells (m21-L).

50. The method of claim 49, wherein the preparing step comprises the steps of:
a. labeling each of the m21 cells with a red cell-tracker dye and each of the m21-L cells with a green cell-tracker dye, respectively;
b. mixing the labeled m21 cells and the labeled m21-L cells to produce a cell mixture;
c. incubating the cell mixture with an anti-CD51 antibody (Ab1) followed by magnetic beads coated with a secondary antibody (Ab2) so as to produce a magnetically-labeled cell sample, wherein Ab2 is specific to Ab1, whereby the magnetic beads are only bound to the m21 cells and free from Ab1; and
d. purifying the magnetically-labeled analyte sample by magnetic bulk separation.

51. The method of claim 50, further comprising the step of quantifying the number of red and green fluorescent cells.

52. The method of claim 48, wherein the magnetically-labeled analyte sample comprises human Th1/Th2 cytokines including interleukin (IL)-2, IL-4, IL-5, IL-6, IL-10, tumor necrosis factor (TNF) and interferon-γ IFN-γ.

53. The method of claim 52, wherein the preparing step comprises the step of incubating magnetic beads with a solution containing the human Th1/Th2 cytokines and secondary fluorescent antibodies.

54. A vector microscope, comprising three orthogonally oriented superconducting quantum interference device (SQUID) sensors.

55. The vector microscope of claim 54, wherein the three SQUID sensors are mounted onto a tip of a sapphire cube, wherein in operation, the sapphire cube is diagonally aligned normal to a scanning plane.

56. The vector microscope of claim 54, wherein the SQUID sensor comprises a directly-coupled low-temperature niobium based SQUID sensor.

57. The vector microscope of claim 54, wherein the SQUID sensor comprises a washer-type SQUID sensor characterized with a SQUID inductance, L, a Josephson junction (JJ) critical current, I_{c}, a JJ self-capacitance, C, and a shunt resistance, Rₙ.

58. The vector microscope of claim 57, wherein the SQUID sensor is adapted such that when the SQUID operates at a temperature of about 4.2 K, the SQUID inductance L, the JJ critical current I_{c}, the JJ self-capacitance C, and the shunt resistance Rₙ satisfy the relationships of β_{c}=2πI_{c}Rₙ²C/φ₀≦0.7 and β_{L}=2LI_{c}/φ₀≅1, wherein φ₀ is a flux quantum of about 2×10⁻¹⁵ Wb.

59. A method of probing the mechanical properties and cell motility of single cells, comprising the steps of:
a. providing a cell sample containing cells and magnetic beads, each magnetic bead attached to a corresponding cell to form a cell-bead unit such that when the magnetic bead moves and/or rotates, the corresponding cell moves and/or rotates accordingly;
b. providing a microfluidic structure defining a detecting zone capable of trapping a single cell therein;
c. providing a vector microscope positioned proximately to the detecting zone of the microfluidic structure, wherein the vector microscope comprises three superconducting quantum interference device (SQUID) sensors orthogonally oriented for simultaneously measuring three orthogonal components of a magnetic field of a cell trapped in the detecting zone;
d. introducing the cell sample into the microfluidic structure, wherein the cell sample is regulated such that each cell-bead unit passes singly through the detecting zone;
e. applying a first magnetic field to the cell sample along a first direction, wherein the first magnetic field comprises a magnetic pulse having an amplitude adapted for saturating magnetic moments of the magnetic beads;
f. applying a second magnetic field to the cell sample along a second direction orthogonally to the first direction, wherein the second magnetic field comprises a uniform magnetic field adapted for creating a torque on the magnetic beads so as to cause them to rotate from a first orientation to a second orientation;
g. turning off the second magnetic field so as to allow the magnetic beads to recover from the second orientation to the first orientation; and
h. continuously measuring a transient magnetic field of the cell-bead unit in the detecting zone in steps (e)-(g), wherein the measured magnetic field is related to the angular rotation of the magnetic bead of the cell-bead unit and hence to the angular rotation of the corresponding cell.

60. The method of claim 59, wherein each magnetic bead is embedded within a corresponding cell through phagocytosis or injection.

61. The method of claim 59, wherein each magnetic bead is bound to the cell membrane of a corresponding cell by coupling of ligand-coated beads of specific cell membrane receptors.

62. The method of claim 59, wherein the angular rotation of the corresponding cell varies time and the strength of the second field, and is related to the mechanical properties and cell motility of the corresponding cell.
